(19) United States
(12) Patent Application Publication
(10) Pub. No.: US 2008/0183264 A1
(43) Pub. Date: Jul. 31, 2008

(54) ELECTRODE CONFIGURATIONS FOR TRANSVASCULAR NERVE STIMULATION

(75) Inventors: Mark J. Bly, Falcon Heights, MN (US); David J. Smith, Shoreview, MN (US); Imad Libbus, St. Paul, MN (US)

Correspondence Address: FAEGRE & BENSON, LLP;32469, 2200 WELLS FARGO CENTER, 90 SOUTH SEVENTH STREET, MINNEAPOLIS, MN, 55402-3901, US

(73) Assignee: CARDIAC PACEMAKERS, INC., St. Paul, MN (US)

(21) Appl. No.: 11/668,957

(22) Filed: Jan. 30, 2007

(51) Int. Cl. A61N 1/05

(52) U.S. Cl. 607/122

(57) ABSTRACT

An intravascular lead adapted to be deployed to a stimulation site within a vessel adjacent a nerve to be stimulated includes at least a first electrode adapted to deliver an electrical pulse across a vessel wall. The first electrode includes an electrically active surface having one or more surface features adapted to focus current. The first electrode is disposed on the distal portion that the electrically active surface can be directed towards the nerve to be stimulated.

### CROSS-REFERENCE TO RELATED APPLICATIONS

This application is related to the following co-pending and co-owned applications entitled: SPIRAL LEAD CONFIGURATIONS FOR INTRAVASCULAR LEAD STABILITY, filed on the same day and assigned Ser. No. ______; DUAL SPIRAL LEAD CONFIGURATIONS, filed on the same day and assigned Ser. No. ______; TRANSVASCULAR LEAD WITH PROXIMAL FORCE RELIEF, filed on the same day and assigned Ser. No. ______; METHOD AND APPARATUS FOR DELIVERING A TRANSVASCULAR LEAD, filed on the same day and assigned Ser. No. ______; METHOD AND APPARATUS FOR DIRECT DELIVERY OF TRANSVASCULAR LEAD, filed on the same day and assigned Ser. No. ______; SIDE PORT LEAD DELIVERY SYSTEM, filed on the same day and assigned Ser. No. ______; and NEUROSTIMULATING LEAD HAVING A STENT-LIKE ANCHOR, filed on the same day and assigned Ser. No. ______, all of which are herein incorporated by reference.

### TECHNICAL FIELD

This application relates to intravascular leads for placement in a vessel adjacent a nerve or muscle to be stimulated. More specifically, the invention relates to intravascular lead electrode configurations for stimulating a nerve from within an adjacent vessel.

### BACKGROUND

A significant amount of research has been directed both to the direct and indirect stimulation of nerves including the left and right vagus nerves, the sympathetic and parasympathetic nerves, the phrenic nerve, the sacral nerve, and the cavernous nerve to treat a wide variety of medical, psychiatric, and neurological disorders or conditions. More recently, stimulation of the vagus nerve has been proposed as a method for treating various heart conditions, including heart failure, tachyarrhythmia, and hypertension.

Typically, in the past, nerve stimulating electrodes were cuffs placed in direct contact with the nerve to be stimulated. A much less invasive approach is to stimulate the nerve through an adjacent vein using an intravascular lead. A lead including one or more electrodes is inserted into a patient's vasculature and delivered at a site within a vessel adjacent a nerve to be stimulated. However, without any additional means of stabilizing the lead within the vein, the lead can move and/or rotate causing the electrodes to migrate from the stimulation site.

Thus, a need exists for an electrode configuration that allows for more control over the stimulation of a nerve, muscle, or tissue from within an adjacent vessel.

### SUMMARY

According to one embodiment of the present invention, an intravascular lead adapted to be deployed to a stimulation site within a vessel adjacent a nerve to be stimulated includes: a lead body including a distal portion and a first and a second conductor in electrical communication with a pulse generator and at least a first electrode coupled to the distal portion and in electrical communication with the first conductor. The first electrode is adapted to deliver an electrical pulse across a vessel wall. According to one embodiment, the first electrode is a cathode including an unmasked electrode portion and a masked electrode portion. The unmasked electrode portion includes a first electrically active surface having one or more surface features adapted to focus current. The first electrode is disposed on the distal portion such that the unmasked portion can be directed towards the nerve to be stimulated. Additionally, the lead also can include at least a second electrode coupled to the distal portion and in electrical communication with the second conductor. The second electrode is adapted to deliver an electrical pulse across a vessel wall. According to one embodiment of the present invention, the second electrode is an anode including a second electrically active surface equal to or greater than the electrically active surface of the first electrode. Like the first electrode, the second electrode is disposed on the distal portion of the lead body such that the electrically active surface can be directed towards the nerve to be stimulated.

According to another embodiment of the present invention, an intravascular lead adapted to be deployed to a stimulation site within a vessel adjacent a nerve to be stimulated includes: a conductive lead body including a proximal end adapted to be connected to a pulse generator; a distal portion comprising at least a first spiral; and an electrode configuration including at least a first electrode. The first electrode is adapted to deliver an electrical pulse across a vessel wall and includes an unmasked electrode portion and a masked electrode portion. The unmasked electrode portion includes a first electrically active surface having one or more surface features adapted to focus current. The first electrode is disposed on the distal portion such that the unmasked portion can be directed towards the nerve to be stimulated.

According to yet another embodiment of the present invention, an intravascular lead adapted to be deployed to a stimulation site within a vessel adjacent a nerve to be stimulated includes: a conductive lead body including a proximal end adapted to be connected to a pulse generator; a distal portion comprising at least a first spiral; and at least a first electrode adapted to deliver an electrical pulse across a vessel wall. The first electrode includes an electrically active surface having one or more surface features adapted to focus current and is disposed on the distal portion such that the electrically active surface can be directed towards the nerve to be stimulated.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a lead according to an embodiment of the present invention deployed within a patient's vascular system.

FIG. 2 is a close-up schematic view of a distal portion of a lead including an electrode configuration according to an embodiment of the present invention deployed within a vessel.

FIGS. 3A and 3B are cross-sectional views of an electrode provided in accordance with an embodiment of the present invention.

FIGS. 4A-4D illustrate the distribution of the current field according to exemplary embodiments of the present invention.

FIGS. 5A-5D are schematic views of an electrode surface provided in accordance with various embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

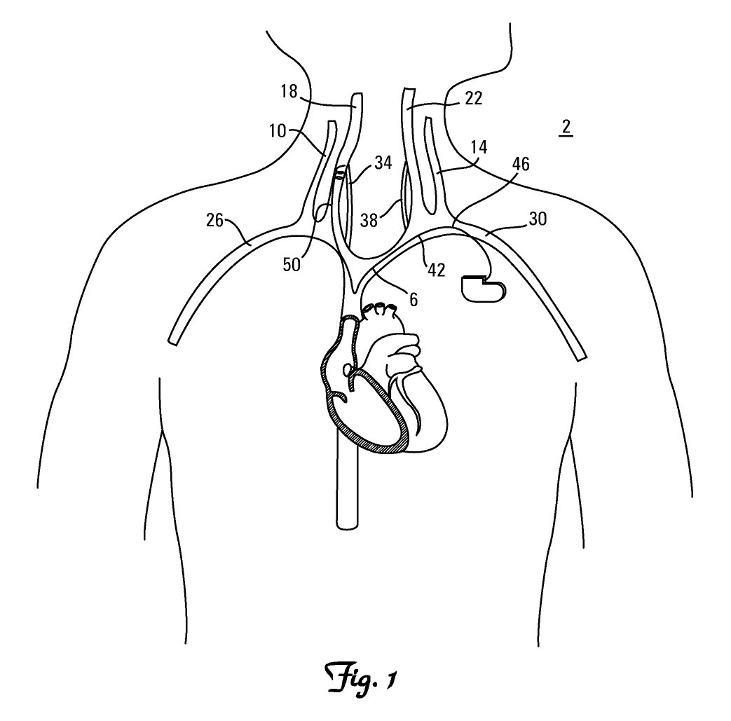

FIG. 1 shows a schematic view of a patient's vascular system 2 showing a lead 6 deployed within the system 2. One or more electrodes 8 are located on the lead 6. In general, the vascular system 2, as shown, includes the right and left external jugular veins 10 and 14, the right and left internal jugular veins 18 and 22, the right and left subclavian veins 26 and 30, portions of which are generally aligned with the right and left vagus nerves 34 and 38. As shown in FIG. 1, the lead 6 is positioned in the right internal jugular vein 18 adjacent to the right vagus nerve 34. It will be appreciated that the lead 6 can be deployed in any vessel adjacent a nerve, muscle, or tissue to be stimulated.

In general, the lead 6 includes a lead body 42 including a proximal portion 46 and a distal portion 50 including one or more electrodes 8. Additionally, the lead 6 includes a proximal end adapted to be connected to a pulse generator or other implantable medical device. The lead body 42 is flexible, and typically has a circular cross-section.

According to one embodiment of the present invention, the lead body 42 includes a plurality of conductors including individual wires, coils, or cables. The conductors can be insulated conductive wires and/or molded in place with an insulator such as silicone, polyurethane, ethylene tetrafluoroethylene, or another biocompatible, insulative polymer. Alternatively, the conductors can be insulated with tubing. In one embodiment of the present invention, the lead body 42 has a co-radial design. In this embodiment, each individual conductor is separately insulated and then wound together in parallel to form a single coil. Alternatively, the lead body 42 is co-axial. According to a further embodiment of the present invention, each conductor is adapted to connect to an individual electrode 8 in a one-to-one manner allowing each electrode 8 to be individually addressable.

According to a further embodiment of the present invention, the distal portion 50 is stiffer than the rest of the lead body 42. One exemplary embodiment of such a structure is disclosed in commonly assigned and co-pending application entitled INTRAVASCULAR LEAD WITH PROXIMAL FORCE RELIEF assigned Ser. No. ______, which is herein incorporated by reference. According to a further embodiment of the present invention, the distal portion 50 includes a material which may impart a desired shape useful for anchoring or securing the distal portion 50 of the lead 6 in a vessel. Exemplary materials include Nitinol and MP35N.

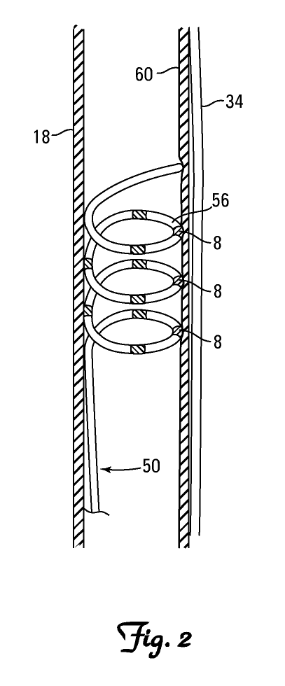

The distal portion 50 can have a variety of configurations adapted to secure and stabilize the lead 6 at a stimulation site located within a vessel 18 adjacent the nerve 34 to be stimulated. For example, as shown in FIG. 2, the distal portion 50 can include a spiral 56. Alternatively, the distal portion 50 can include more than one spiral. In embodiments including two or more spirals, the spirals can be interrupted by a generally straight portion. Exemplary distal portions 50 including spirals are shown and described in commonly assigned and co-pending application Ser. No. ______, entitled SPIRAL CONFIGURATIONS FOR INTRAVASCULAR LEAD STABILITY and in commonly assigned and co-pending application Ser. No. ______, entitled DUAL SPIRAL LEAD CONFIGURATIONS, both of which are herein incorporated by reference. An alternative exemplary distal portion is shown and described in commonly assigned and co-pending application Ser. No. ______, entitled NEUROSTIMULATING LEAD HAVING A STENT-LIKE ANCHOR, also which is herein incorporated by reference.

The distal portion 50 of the lead 6 also includes a plurality of electrodes 8 forming an electrode configuration. According to one exemplary embodiment, at least one electrode 8 is adapted to function as a cathode, and at least one electrode 8 is adapted to function as an anode. The electrodes 8 are located on the distal portion 50 of the lead body 42 such that they can be directed towards the adjacent nerve, muscle, or tissue to be stimulated. According to one exemplary embodiment, the electrodes 8 can be located on at least one spiral, a straight portion interrupting two or more spirals, and/or a combination thereof. This increases the potential for at least one electrode 8 to deliver an electrical stimulus across a vessel wall 60 to the adjacent nerve, muscle, or tissue.

In one exemplary embodiment of the present invention, shown in FIG. 2, the electrodes 8 are located on a spiral 56. According to the embodiment shown in FIG. 2, the electrodes 8 are pressed up against the vessel wall 60 maximizing electrical transvascular stimulation when the spiral 56 is expanded. According to a further embodiment of the present invention, the spiral 56 presses up against the vessel wall 60 with enough radial expansion force such that the turns of the spiral 56 migrate outside of the original boundaries of the vessel wall 60 and towards the nerve 34 to be stimulated without damaging the vessel wall 60. As a result, any electrodes 8 located on the spiral 56 are placed in closer proximity to the nerve 34.

Multiple electrodes 8 allow flexibility in the intravascular placement of the distal portion 50 of the lead 6. Not all of the electrodes 8 need to be directed towards the adjacent nerve or muscle tissue in order for maximum stimulation across the vessel wall to occur. Likewise, the circular or elliptical cross section of the spirals allow the distal portion 50 of the lead 6 to be rotated within a vessel so as to ensure that at least one set of electrodes 8 is capable of delivering a sufficient electrical stimulating pulse across the vessel wall.

According to a further embodiment of the present invention, the electrodes 8 are spaced an equal distance from one another. In one embodiment, the electrodes 8 are spaced a distance of about 2 to about 20 millimeters from one another. According to a further embodiment, the electrodes 8 are spaced a distance of about 3 to about 11 millimeters from one another. At least one electrode 8 is adapted to deliver an electrical pulse transvascularly to a nerve, muscle, or tissue to be stimulated from within the adjacent vessel in which the distal portion 50 of the lead 6 is deployed.

According to one embodiment of the present invention, the electrodes 8 are connected to multiple individual conductors extending through the lead body 42 allowing for them to be individually addressable. The electrodes 8 are electrically coupled in a one-to-one relationship to individual conductors located within the lead body 42 and the distal portion 50. The conductors are adapted to deliver an electrical signal from a pulse generator to the electrodes 8. Individually addressable electrodes are capable of producing stimulation patterns along the distal portion 50 of the lead body 42. Individually addressable electrodes allow for flexibility in electrode selection and control over the direction of stimulation allowing for multiple options for stimulation and sensing.

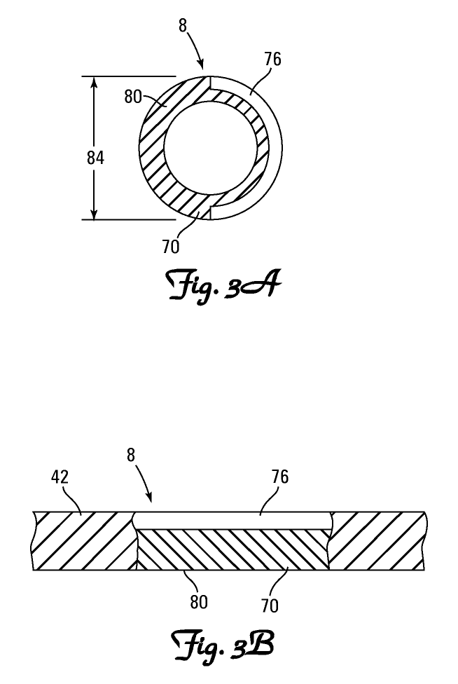

FIGS. 3A and 3B are cross-sectional views of an electrode according to an embodiment of the present invention. As shown in FIGS. 3A-3B, one or more electrodes 8 includes an unmasked portion 70, a masked portion 76, and an electrically active surface area 80. The electrodes 8 are typically made of a corrosive-resistant conductive material. Exemplary materials include, but are not limited to, the following: platinum; iridium; platinum-iridium; and combinations thereof. The electrodes may be deposited on a support layer formed in the lead body. According to a further embodiment of the present invention, the electrically active surface area 80 is coated with iridium oxide (IROX) or another oxide layer to increase sensitivity of the electrode and increase impedance. Platinum black can also be used to coat the electrically active surface 80 of the electrode 8 to increase the amount of electrically active surface area 80.

The unmasked portion 70 of the electrode 8 is adapted to focus current towards the area to be stimulated. In one exemplary embodiment, the unmasked portion 70 forms an arc 84 ranging from about 45 to about 200 degrees and includes an electrically active surface 80 having a surface area ranging from about 1 to about 20 mm². Additionally, the electrode 8 can extend from about less than 1 to about 10 millimeters along a spiral, such as spiral 56 shown in FIG. 2. According to one embodiment of the present invention, the masked portion 76 can be masked by the outer insulation of the lead body 42. According to this embodiment, the outer insulation is slit or cut away to form the unmasked portion 70 revealing the electrically active surface 80. In an alternate embodiment, a silicone or other medical adhesive forms the masked portion 76 of the electrode 8. Additionally, the masked portion 76 can be formed by molded biocompatible polymers or other insulative materials known to those of skill in the art.

According to one exemplary embodiment, the electrodes 8 are located on an outer circumference of a spiral, such as spiral 56 shown in FIG. 2, such that the unmasked portion 70 including its electrically active surface 80 is directed towards the adjacent nerve, muscle, or tissue to be stimulated. According to an alternative exemplary embodiment, the electrodes 8 are located on an outer surface of a distal portion of a lead mounted over a stent-like anchor. The masked portion 76 is located on an inner circumference of the spiral or distal portion and aids in shielding other muscles or innervated areas of the patient's anatomy from being stimulated when stimulation of those areas is not desired.

According to yet a further embodiment of the present invention, the distal portion 50 includes a first electrode 8 acting as a cathode 8 and a second electrode 8 acting as an anode. The cathode includes an unmasked portion 70 and a masked portion 76. The anode, like the cathode, also includes an unmasked portion 70 and a masked portion 76. In this embodiment the arc length 84 and electrically active surface area of the anode is greater than that of the cathode. According to another embodiment of the present invention, the anode is a ring or a partial ring electrode whose electrically active surface area is greater than that of the cathode. According to one exemplary embodiment, the anode has an electrically active surface area ranging from about 6 to about 12 mm² and the cathode has an electrically active surface area ranging from about 3 to about 6 mm².

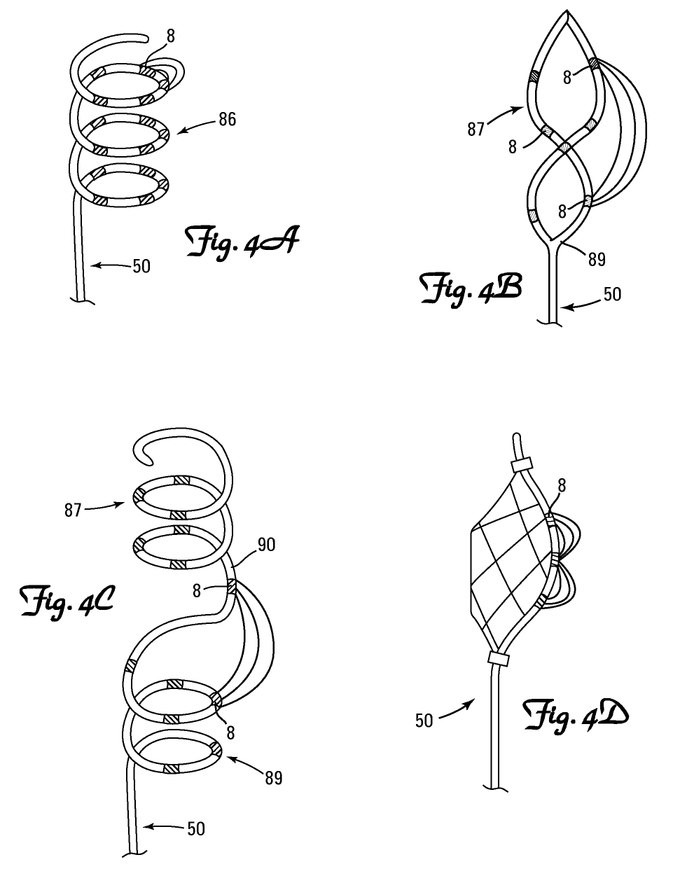

FIGS. 4A-4D show the distribution of current field densities according to exemplary embodiments of the present invention. The ability to stimulate between electrodes 8 in different locations on the lead body 42 provides control over the distribution of the current field. The current field distribution can be limited and thus the region narrowed in which the field is strong enough to stimulate, resulting in a more focused stimulation. Additionally, the ability to selectively stimulate between multiple electrodes 8 can aid in shielding other muscles or innervated areas of a patient's anatomy from being stimulated when stimulation of those areas is not desired. For example, stimulation can occur between electrodes on the same turn of the spiral 86 to produce a narrow distribution of the current field (shown in FIG. 4A), between electrodes 8 located on different spirals 87, 89 (as shown in FIG. 4B), and between electrodes 8 located on a spiral 89 and a generally straight portion 90 (as shown in FIG. 4C). According to another exemplary embodiment (shown in FIG. 4D), tripolar stimulation can also occur.

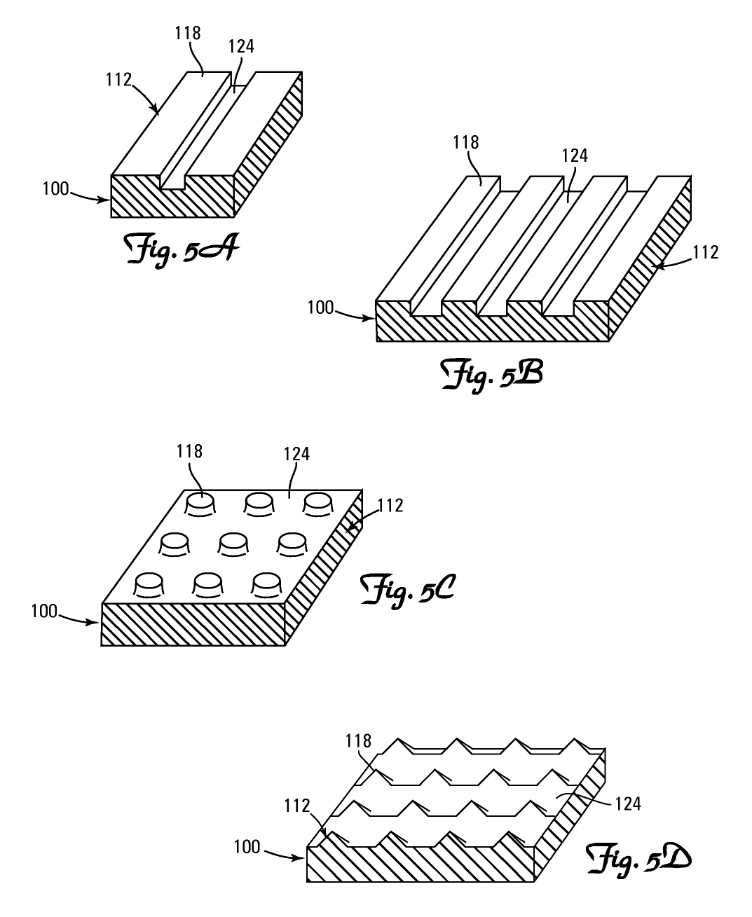

FIGS. 5A-5D are schematic views of electrically active electrode surfaces 80 according to various embodiments of the present invention. As shown in FIGS. 5A-5D, the electrically active surface 80 includes one or more surface features 112. The surface features 112 are adapted to concentrate or focus current towards the adjacent nerve, muscle or tissue to be stimulated. The surface features 112 can include nubs or other protrusions that extend from the electrode surface 80 towards the stimulation target. The surface features 112 are configured such that current is focused at their edges. According to one embodiment, the nubs or protrusions are configured such that current is focused at their tip. The surface features 112 also can be used to force the electrode 8 into greater intimate contact with the vessel wall, thus placing the electrically active surface 80 closer to the nerve or muscle over a localized area to achieve a lower stimulation threshold or achieve a better sensing capability.

As shown in FIGS. 5A-5D, when multiple surface features 112 are provided, the surface features 112 can form a pattern on the electrically active surface 80. According to one embodiment of the present invention, the pattern can include an electrically conductive surface 118 and an insulative surface 124. According to a further embodiment of the present invention, the electrically conductive surface 118 is elevated with respect to the insulative surface 124. According to an alternate embodiment, the electrically conductive surface 118, is recessed with respect to the insulative surface 124. In one exemplary embodiment, as shown in FIG. 5A, and the surface features 112 include two or more raised ridges that are generally in parallel alignment with one another. According to another embodiment of the present invention, as shown in FIG. 5C, the surface features 112 are a series of raised nubs, cylinders, or other similarly shaped protrusions. In an alternate embodiment, the surface features 112 shown in FIG. 5C can be depressed into the electrode surface. Finally, as shown in FIG. 5D, the surface features 112 can be a plurality of peak-like protrusions extending away from the electrode surface.

According to a further embodiment of the present invention, the nubs or protrusions, as shown in FIGS. 5C and 5D, are adapted to pierce the vessel wall. In exemplary embodiment, the nubs or protrusions forming the surface features 112 pierce the vessel wall and are in direct contact with the nerve to be stimulated. In another exemplary embodiment, the nubs or protrusions are at a distance of about less than 2 mm from the nerve to be stimulated.

According to another embodiment of the present invention, the electrode 8 is a conductive polymer patterned electrode as described in co-owned and co-pending U.S. application Ser. No. ______, which is herein incorporated by reference. According to yet another embodiment of the present invention, the electrode 8 also includes a drug eluting collar as described in U.S. Pat. No. 6,889,092, also which is herein incorporated by reference.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. An intravascular lead adapted to be deployed to a stimulation site within a vessel adjacent a nerve to be stimulated, the lead comprising:
a lead body including a distal portion and a first and a second conductor in electrical communication with a pulse generator;
at least a first electrode coupled to the distal portion and in electrical communication with the first conductor, the first electrode adapted to deliver an electrical pulse across a vessel wall, wherein the first electrode is a cathode including an unmasked electrode portion and a masked electrode portion, the unmasked electrode portion comprising a first electrically active surface including one or more surface features adapted to focus current, and wherein the first electrode is disposed on the distal portion such that the unmasked portion can be directed towards the nerve to be stimulated; and
at least a second electrode coupled to the distal portion and in electrical communication with the second conductor, the second electrode adapted to deliver an electrical pulse across a vessel wall, wherein the second electrode is an anode including a second electrically active surface equal to or greater than the electrically active surface of the first electrode, and wherein the second electrode is also disposed on the distal portion of the lead body such that the electrically active surface can be directed towards the nerve to be stimulated.

2. The intravascular lead according to claim 1, wherein the unmasked portion of the first electrode comprises an electrically active surface forming an arc ranging from about 45 to 200 degrees.

3. The intravascular lead according to claim 1, wherein the second electrode comprises an unmasked portion and a masked portion, the unmasked portion of the electrode comprising an electrically active surface forming an arc ranging from about 45 to about 200 degrees, wherein the arc of the second electrode is equal to or greater than an arc of the unmasked portion of the first electrode.

4. The intravascular lead according to claim 1, wherein the first electrode comprises an electrically active surface area ranging from about 1 to about 20 mm², and the second electrode comprises an electrically active surface area that is equal to or greater than the electrically active surface area of the first electrode.

5. (canceled)

6. The intravascular lead according to claim 1, wherein the nerve is the vagus nerve and the vessel is selected from the group consisting of an internal jugular vein, a superior vena cava, and a brachiocephalic vein.

7. The intravascular lead according to claim 1, wherein the one or more surface features are protrusions formed on the electrically active surface adapted to pierce a vessel wall.

8. (canceled)

9. The intravascular lead according to claim 1, wherein the second electrode includes one or more surface features adapted to focus current.

10. The intravascular lead according to claim 9, wherein the one or more surface features are protrusions formed on the electrically active surface adapted to pierce a vessel wall-forming a pattern.

11. (canceled)

12. An intravascular lead adapted to be deployed to a stimulation site within a vessel adjacent a nerve to be stimulated, the lead comprising:
a conductive lead body including a proximal end adapted to be connected to a pulse generator;
a distal portion comprising at least a first spiral; and
an electrode configuration including at least a first electrode adapted to deliver an electrical pulse across a vessel wall, the first electrode including an unmasked electrode portion and a masked electrode portion, the unmasked electrode portion comprising a first electrically active surface including one or more surface features adapted to focus current, and wherein the first electrode is disposed on the distal portion such that the unmasked portion can be directed towards the nerve to be stimulated.

13. The intravascular lead according to claim 12, wherein the electrode configuration further comprises a second electrode having an electrically active surface and adapted to deliver an electrical pulse across a vessel wall, wherein the second electrode is located on the distal portion of the lead body such that the electrically active surface can be directed towards the nerve to be stimulated.

14. The intravascular lead according to claim 13, wherein the first electrode is a cathode and the second electrode is an anode, wherein the electrically active surface of the second electrode is greater than the electrically active surface of the first electrode.

15. The intravascular lead according to claim 13, wherein the first and second electrodes are located adjacent to one another on the distal portion.

16. The intravascular lead according to claim 13, wherein the distal portion further includes a second spiral, wherein the first and second electrode can be located on the first spiral, the second spiral, or both the first and second spirals.

17. The intravascular lead according to claim 13, wherein the distal portion further includes a second spiral and a generally straight portion, wherein the first and second electrodes can be located on the first spiral, the second spiral, the generally straight portion, or a combination thereof.

18. The intravascular lead according to claim 12, wherein the unmasked portion of the first electrode comprises an electrically active surface forming an arc ranging from about 45 to 200 degrees.

19. The intravascular lead according to claim 13, wherein the second electrode comprises an unmasked portion and a masked portion, the unmasked portion of the electrode comprising an electrically active surface forming an arc ranging from about 45 to about 200 degrees, wherein the arc of the second electrode is equal to or greater than an arc of the unmasked portion of the first electrode.

20. The intravascular lead according to claim 12, wherein the nerve is the vagus nerve and the vessel is selected from the group consisting of an internal jugular vein, a superior vena cava, and a brachiocephalic vein.

21. The intravascular lead according to claim 12, wherein the first electrode comprises a plurality of surface features forming a pattern.

22. An intravascular lead adapted to be deployed to a stimulation site within a vessel adjacent a nerve to be stimulated, the lead comprising:
a conductive lead body including a proximal end adapted to be connected to a pulse generator;
a distal portion comprising at least a first spiral; and
at least a first electrode adapted to deliver an electrical pulse across a vessel wall, wherein the first electrode includes an electrically active surface including one or more surface features adapted to focus current, wherein the first electrode is disposed on the distal portion such that the electrically active surface can be directed towards the nerve to be stimulated.

23. The intravascular lead according to claim 22, further comprising at least a second electrode adapted to deliver an electrical pulse across a vessel wall, wherein the second electrode includes a second electrically active surface larger than the electrically active surface of the first electrode, and wherein the second electrode is also disposed on the distal portion of the lead body such that the electrically active surface can be directed towards the nerve to be stimulated.

24. (canceled)

25. The intravascular lead according to claim 22, wherein the one or more surface features are protrusions adapted to pierce a vessel wall.

26. The intravascular lead according to claim 23, wherein the first electrode further comprises an unmasked portion and a masked portion, the unmasked portion of the first electrode having an electrically active surface forming an arc ranging from about 45 to 200 degrees.

27. The intravascular lead according to claim 26, wherein the second electrode comprises an unmasked portion and a masked portion, the unmasked portion of the second electrode comprising an electrically active surface forming an arc ranging from about 45 to about 200 degrees, wherein the arc of the second electrode is equal to or greater than the arc of the unmasked portion of the first electrode.

28. The intravascular lead according to claim 23, wherein the first electrode comprises an electrically active surface area ranging from about 1 to about 20 mm², and wherein the second electrode comprises an electrically active surface area from about 1 to about 20 mm², wherein the electrically active surface area of the second electrode is greater than the electrically active surface area of the first electrode.

29. The intravascular lead electrode configuration according to claim 22, where the vessel is selected from the group consisting of an internal jugular vein, a superior vena cava, and a brachiocephalic vein, and wherein the nerve is the vagus nerve.
